(19) United States
(12) Patent Application Publication
(10) Pub. No.: US 2023/0301954 A1
(43) Pub. Date: Sep. 28, 2023

(54) Method of Metabolic Enhancement and Composition Manufacture

(71) Applicant: David Michalski, Alexandria, VA (US)

(72) Inventor: David Michalski, Alexandria, VA (US)

(21) Appl. No.: 18/113,842

(22) Filed: Feb. 24, 2023

Related U.S. Application Data
(60) Provisional application No. 63/314,055, filed on Feb. 25, 2022.

(51) Int. Cl. A61K 31/198; A61K 31/7084; A61K 31/205; A61K 9/00; A61K 9/48

(52) U.S. Cl. CPC A61K 31/198; A61K 9/0053; A61K 9/48; A61K 31/205; A61K 31/7084

(57) ABSTRACT

A method of enhancing and speeding a person’s metabolic activity and related method of manufacturing a substance for such enhancement.

### CROSS REFERENCE TO RELATED APPLICATIONS

Not applicable.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

### Field of the Invention

The invention is a method of enhancing metabolic activity in a person.

### DETAILED DESCRIPTION OF THE INVENTION

The invention constitutes a means of enhancing and speeding a person’s metabolic activity and related means of preparing a substance for such enhancement. Supplements for the adjustment of metabolic activity are known, and the following supplements are particularly well-regarded for enhancing and speeding the human metabolism of nutrients. Each of these substances plays a role in metabolizing nutrients for the production of ATP, which is used to power cellular activity.

L-Carnitine Tartrate is a “fat burner” diet supplement used to prevent and treat low blood levels of carnitine. Carnitine is a substance made in the body from meat and dairy products, which helps the human body metabolize long-chain fatty acids for quick energy. It also plays a detoxifying role by ferrying incompletely oxidized fatty acids and organic acids out of cell mitochondria, where they can be excreted. Studies support its use for health, brain function and disease prevention. L-Carnitine Tartrate can be taken in powder, pill or liquid form.

N-Acetyl Cysteine (NAC) is known for antioxidant production and, in combination with glutamine and glycine, is necessary to make and replenish glutathione. Glutathione is an antioxidant compound that neutralizes free radicals (H⁺) that damage body cells. NAC also in detoxification of certain chemicals in the body, breaks up mucus and helps regulate the levels of brain neurotransmitter glutamate.

NAD⁺ (nicotinamide adenine dinucleotide) is an essential coenzyme used in cellular mitochondria. It is both key in the conversion of food to energy and in maintaining DNA integrity to ensure proper cell function. NAD⁺ transfers electrons from one molecule to another within cells to carry out necessary reactions, including metabolic reactions.

L-Serine is a polar amino acid used in the biosynthesis of proteins. It is important in human metabolism in that it participates in the biosynthesis of purines and pyrimidines. It is the precursor to several amino acids including glycine and cysteine. It is also the precursor to numerous other metabolites, including sphingolipids and folate, which is the principal donor of one-carbon fragments in biosynthesis.

While these substances are manufactured by the human body, production levels vary substantially person to person. Additionally, production levels can decrease in older, ill and overweight people, slowing metabolism with the result of lower energy levels and greater weight retention. For some people, the synergetic effect of lower metabolism and higher body weight can become cyclical.

The inventive concept is the administration of periodic doses of each of the four discussed supplement substances, combined to form a single composition, to generate a measurable increase in a person’s metabolic rate. Periods of dosing can be daily, every other day, semiweekly or weekly. Daily dosing is preferred. Specifically, a combination of the components has been found to be effective in the following amounts: 65-75% L-Carnitine Tartrate, 15-25% N-Acetyl Cysteine, 5-15% NAD⁺ and 5-15% L-Serine. In a more preferred embodiment, the percentages are: 68-70% L-Carnitine Tartrate, 19-22% N-Acetyl Cysteine, 9-11% NAD⁺ and 9-11% L-Serine. Variations in the percentages deemed effective for metabolic enhancement may vary further to account for differences in the age, size, fitness, lifestyle and other physical characteristics of a given user.

The administration of the composition would be through any known method, including the use of pills, gelcaps, liquid, etc. Oral administration is preferred.

In a preferred embodiment, the daily dosage for an adult person would be 2900 mg, with approximately half such dosage for an adolescent person.

The manufacture of the composition would be accomplished by mixing, preferably of dry powder or granular stock of each supplement, using standard pharmacological laboratory equipment. An alternative embodiment would be the mixing of liquid varieties of the supplements and administration of the composition in liquid form.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable equivalents.

I claim:

1. A method of enhancing metabolic activity in a person, comprising the step of administering a composition to a person, wherein the composition comprises a mixture of:
L-Carnitine Tartrate,
N-Acetyl Cysteine,
NAD⁺, and
L-Serine.

2. The method of claim 1, wherein the components are mixed in the following percentage ranges by total weight:
L-Carnitine Tartrate, 65-75%,
N-Acetyl Cysteine, 15-25%,
NAD⁺, 5-15% and
L-Serine, 5-15%.

3. The method of claim 1, wherein the components are mixed in the following percentage ranges by total weight:
L-Carnitine Tartrate, 68-70%,
N-Acetyl Cysteine, 19-22%,
NAD⁺, 9-11% and
L-Serine, 9-11%.

4. A method of manufacturing the components of claim 1, comprising:
1. Obtaining premade dietary supplement stock components comprising L-Carnitine Tartrate, N-Acetyl Cysteine, NAD⁺, and L-Serine;
2. Mixing the components into a single composition using the following percentages by total weight:
L-Carnitine Tartrate, 65-75%,
N-Acetyl Cysteine, 15-25%,
NAD⁺, 5-15% and
L-Serine, 5-15%, and
3. Loading the mixed components into gelcaps of approximately 2900 mg each.

5. The method of manufacture of claim 4, wherein the dietary supplement stock components are in a dry, granular state.

6. The method of manufacture of claim 5, wherein the mixing is accomplished with a lab powder mixer, agitator, tumbling mixer, planetary mixer, nauta mixer ribbon mixer or similar known equipment.

7. The method of manufacture of claim 4, wherein the dietary supplement stock components are in a dry powder state, adding the supplemental step 3:
3. Applying granulation liquid to the composition.

8. The method of manufacture of claim 7, wherein steps 2 and 3 are accomplished with a fluidized bed granulator system.
